(19) United States
(12) Patent Application Publication
(10) Pub. No.: US 2009/0110701 A1
(43) Pub. Date: Apr. 30, 2009

(54) T-CELL MODULATION

(75) Inventors: Frank Ward, Aberdeen (GB); Robert Barker, Aberdeen (GB)

Correspondence Address: SPECKMAN LAW GROUP PLLC, 1201 THIRD AVENUE, SUITE 330, SEATTLE, WA, 98101, US

(73) Assignee: THE UNIVERSITY COURT OF THE UNIVERSITY OF ABERDEEN, Aberdeen (GB)

(21) Appl. No.: 11/720,834

(22) PCT Filed: Dec. 2, 2005
(86) PCT No.: PCT/GB05/04639

§ 371 Date: Jun. 4, 2007

(30) Foreign Application Priority Data
Dec. 3, 2004 (GB) 0426644.1

(51) Int. Cl. A61K 39/00; C12N 5/06

(52) U.S. Cl. 424/275.1; 435/375; 424/184.1

(57) ABSTRACT

The invention provides methods and materials for use in modulating T cell activation, based on the production and secretion of soluble cytotoxic T-lymphocyte antigen-4 (sCTLA-4) by cells of the immune system. The method involves stimulating secretion of endogenous sCTLA-4 by T cells, which have preferably previously been exposed to an antigen, by exposing the said cells to a stimulatory agent, preferably a peptide which comprises at least one antigenic determinant of said antigen. The cells may also be exposed to a CD28 stimulatory binding agent, either alone or in combination with the antigenic peptide. In preferred embodiments, the method may be used for treatment or prophylaxis of a disease characterized by a pathogenic immune or autoimmune response. The invention also provides a system for inhibiting sCTLA-4 secretion by T cells.

### TECHNICAL FIELD

The present invention relates generally to methods and materials for use in modulating T cell activation, based on the production and secretion of soluble CTLA-4 (sCTLA-4) by cells of the immune system.

### BACKGROUND ART

T-cells form a crucial element of the adaptive immune response but their powerful effects need to be assiduously managed by the immune system to avoid undesirable destructive responses to self tissues. Successful T-cell activation requires 2 signals, T cell receptor (TCR) encounter with a specific peptide ligand bound to MHC, and costimulation, a process mediated by, for example, ligation of the T-cell membrane protein CD28 with B7.1/B7.2 (CD80/CD86) molecules on professional APC (FIG. 1a).

Activated T-cells later express a shared sequence homologue of CD28, CTLA-4, which competes with CD28 for B7.1/B7.2 ligation and restores activated T-cells to their resting state (FIG. 1b)(Brunet J F, Denizot F, Luciani M F, Roux-Dosseto M, Suzan M, Mattei M G and Golstein P. (1987) A new member of the immunoglobulin superfamily—CTLA-4. Nature 328(6127): 267-270; Walunas T L, Lenschow D J, Bakker C Y, Linsley P S, Freeman G J, Green J M, Thompson C B and Bluestone J A. (1994) CTLA-4 can function as a negative regulator of T cell activation. Immunity 1(5): 405-413; Kearney E R, Walunas T L, Karr R W, Morton P A, Loh D Y, Bluestone J A and Jenkins M K. (1995) Antigen-dependent clonal expansion of a trace population of antigen-specific CD4+T cells in vivo is dependent on CD28 costimulation and inhibited by CTLA-4. J Immunol. 155(3): 1032-1036). The importance of CTLA-4 in T-cell regulation was demonstrated by CTLA-4 knockout mice which die shortly after birth because they lack the ability to regulate T-cell activation and expansion (Tivol E A, Borriello F, Schweitzer A N, Lynch W P, Bluestone J A and Sharpe A H. (1995) Loss of CTLA-4 leads to massive lymphoproliferation and fatal multiorgan tissue destruction, revealing a critical negative regulatory role df CTLA-4. Immunity 3(5): 541-547).

Further, blockade of CTLA-4 function with anti-CTLA-4 antibody can promote effective anti-tumor responses.

Soluble cytotoxic T-lymphocyte antigen-4 (sCTLA-4) is a 652-bp alternative transcript of full length membrane bound CTLA-4 (mCTLA-4). mCTLA-4 shares its extracellular domain with sCTLA-4 but the entire transmembrane domain has been deleted. This deletion also results in a reading frame change which renders what would have been the cytoplasmic domain of mCTLA-4 as vestigial sequence on sCTLA-4 and with no predicted function. Thus sCTLA-4 can be secreted and can bind B7.1/B7.2 with the same high affinity as mCTLA-4 but is predicted to have no other function. sCTLA-4 transcripts have also been identified in mouse and rat as well as human. By blocking CD28 from binding B7.1/B7.2, sCTLA-4 can very effectively inhibits T-cell responses (i.e. sCTLA-4 prevents and suppresses T-cell activation) (see Magistrelli, G., Jeannin, P., Herbault, N., decoignac, A. B., Gauchat, J-F., Bonnefoy, J-Y., and Deineste, Y. (1999) A soluble form of CTLA-4 generated by alternative splicing is expressed by nonstimulated human T cells. Eur. J. Immunol. 29: 3596-3602; Oaks, M. K., Hallett, K. M., Penwell, R. T., Stauber, E. C., Warren, S. J., and Tector, A. J. (2000) A native soluble form of CTLA-4. Cell. Immunol. 201: 144-153.)

Recently, an extensive genetic mapping study identified single nucleotide polymorphisms (SNP) in the untranslated region 3′ of the CTLA-4 gene, which correlated with low efficiency of sCTLA-4 expression (Ueda, H., Howson, J. M., Esposito, L., Heward, J., Snook, H., et al. (2003) Association of the T-cell regulatory gene CTLA4 with susceptibility to autoimmune disease. Nature advance online publication, 30 Apr. 2003 (doi: 10.1038/nature01621).

Thus low sCTLA-4 secretion may be a contributory factor towards susceptibility of certain individuals to autoimmune disease. However, serum levels of sCTLA-4 were increased in patients with autoimmune thyroid disease (Oaks, M. K., Hallett, K. M., Penwell, R. T., Stauber, E. C., Warren, S. J., and Tector, A. J. (2000) A native soluble form of CTLA-4. Cell. Immunol. 201: 144-153.), systemic lupus erythematosus, and active myasthenia gravis, compared with normal volunteers (Wang, X-B., Kakoulidou, M., Giscombe, R., Qiu, Q., Huang, D R., Pirskanen, R., and Lefvert, A. K. (2002) Abnormal expression of CTLA-4 by T-cells from patients with myasthenia gravis: effect of an AT-rich gene sequence. J. Neuroimmunol. 130: 224-232; Liu M F, Wang C R, Chen P C and Fung L L. (2003) Increased expression of soluble cytotoxic T-lymphocyte-associated antigen-4 molecule in patients with systemic lupus erythematosus. Scand J Immunol. 57(6): 568-572). Further, in a recent study, increased sCTLA-4 corresponded well with active diffuse systemic sclerosis (Sato S, Fujimoto M, Hasegawa M, Komura K, Yanaba K, Hayakawa I, Matsushita T and Takehara K. (2004) Serum soluble CTLA-4 levels are increased in diffuse cutaneous systemic sclerosis. Rheumatology (Oxford) 43(10): 1261-1266).

However, reports of sCTLA-4 in the literature have shown that it is secreted by resting rather than activated T-cells (Magistrelli supra; Oaks supra). Indeed, powerful non-specific activation of T-cells by mitogens or anti-CD3 antibodies results in a reduction in sCTLA-4 secretion.

Therefore CTLA-4 therapies to date have generally focused on synthetic recombinant form of CTLA-4 (e.g. CTLA4-Ig) which is currently being evaluated as a therapy for autoimmune diseases in a number of clinical trials (Lenschow D J, Zeng Y, Thistlethwaite J R, Montag A, Brady W, Gibson M G, Linsley P S and Bluestone J A. Long-term survival of xenogeneic pancreatic islet grafts induced by CTLA4Ig. Science 257(5071): 789-792; Vincenti F. (2002) What's in the pipeline ? New immunosuppressive drugs in transplantation. Am J Transplant. 2(10): 898-903; Emery P. (2003) The therapeutic potential of costimulatory blockade with CTLA4Ig in rheumatoid arthritis. Expert Opin Investig Drugs 12(4): 673-681).

Nevertheless it can be seen that novel methods of utilizing sCTLA-4 would provide a contribution to the art.

WO97/20574 discusses the methods and compositions for increasing the activation of T cells through a blockade of CTLA-4 signaling.

U.S. Patent No. 6,107,056 (Oaks) is concerned with the SCTLA-4 gene and product

U.S. Pat. No. 6,337,316 (El Tayar) is concerned with peptidomimetics said to be capable of inhibiting CD28 and/or CTLA-4 interaction with CD80 (B7-1) and CD86 (B7-2) and having a particular core amino acid sequence.

### DISCLOSURE OF THE INVENTION

The present inventors have found that T-cells which respond specifically to peptides presented on MHC Class II can be induced to secrete sCTLA-4. For example, and as discussed in more detail below, FIG. 2 shows antigen-specific Th2 responses corresponded with an increase in sCTLA-4. It was also shown that PBMC from a patient with Autoimmune hemolytic anemia (AIHA—a Th1 mediated autoimmune disease) responded specifically to the AIHA-associated autoantigen RhD autoantigen by secreting higher levels of sCTLA-4 and IL-4 compared with negative controls (FIG. 3). FIG. 2 also shows secretion of sCTLA-4 by stimulated cells, using alternative agents.

There have been no previous reports in the literature which suggest that immune system cells, specifically T-cells, can be activated and induced to secrete sCTLA-4. Therefore the inventors have shown, for the first time, that sCTLA-4 secretion, a powerful inhibitor of T-cells, can be artificially induced by applying specific antigens to T-cells.

Thus in one aspect, the invention relates generally to methods of stimulating sCTLA-4 secretion by T cells (which may be isolated cells) which method comprises exposing said cells to a stimulatory agent such as to induce secretion of endogenous sCTLA-4 therefrom.

In such methods sCTLA-4 may be used to inter alia increase tolerance to a particular target antigen, thereby helping avoid or mitigate a destructive anti-self response, as has been suggested for recombinant CTLA4-Ig (see e.g. Kremer J M, Westhovens R, Leon M, Di Giorgio E, Alten R, Steinfeld S, Russell A, Dougados M, Emery P, Nuamah I F, Williams G R, Becker J C, Hagerty D T and Moreland L W. (2003) Treatment of rheumatoid arthritis by selective inhibition of T-cell activation with fusion protein CTLA4-Ig. N Engl J Med. 349(20): 1907-1915; Wallace P M, Rodgers J N, Leytze G M, Johnson J S and Linsley P S. (1995) Induction and reversal of long-lived specific unresponsiveness to a T-dependent antigen following CTLA4-Ig treatment. J Immunol. 154(11): 5885-5895).

Other aspects of the invention provide for novel agents, assays, methods and treatments based on the above observations.

Some aspects of the invention will now be discussed in more detail.

In one aspect the invention provides a method of stimulating sCTLA-4 secretion by T cells which have previously been exposed to an antigen, which method comprises exposing said cells to an agent which stimulates endogenous secretion of sCTLA-4 therefrom, which agent is a peptide comprising at least one antigenic determinant of said antigen.

In one embodiment the method may comprise exposing the cells to an agent which comprises both a peptide comprising at least one antigenic determinant of said antigen, and also a CD28 stimulatory binding agent. As demonstrated below, the inventors have shown that in such (combination) agents, a CD28 stimulatory binding agent can augment the sCTLA secretion caused by the peptide which includes the antigenic determinant. The agents of the invention may thus comprise or provide these separate components in combination (e.g. simultaneously). Accordingly it will be understood that in any of the following aspects or embodiments, a CD28 stimulatory binding agent may optionally also be present.

As further discussed below, the antigen may be associated with the pathogenic immune or autoimmune response—either derived from the precise antigen targeted by pathogenic T-cells or from bystander self tissues damaged in consequence of that response.

In preferred embodiments methods described above thereby inhibit the activity or activation of a T cell in response to a previously-encountered antigen.

The agent (which is preferably the agent which comprises a peptide comprising at least one antigenic determinant of said antigen) may preferentially or selectively stimulates secretion of sCTLA-4 relative to a pathogenic or otherwise undesirable T-cell activity—for example relative to other antigen-specific T-cell mediated responses such as delayed type hypersensitivity. Such responses may include proliferation, differentiation, and various effector functions leading to inflammation, or CTL responses. In particular the agent stimulates secretion of sCTLA-4 relative to release of cytokines cytokines associated with expansion of Th1/Th2 T cell subsets in immune-mediated disorders including Interferon-γ, TNF-α, IL-12, IL-4, IL-5, IL-13, and IL-18.

T Cells

As discussed in more detail below, the method may be performed in vivo or in vitro, and in particular may be performed in the context of a population of cells, of which the T cells are a part. Naturally the in vivo environment will include a population of cells in any case.

Such T cells may for example be CD4⁺ and\or CD8⁺ T lymphocytes. Preferably the T cells comprises at least CD4⁺ T lymphocytes and at least one type of antigen presenting cell (APC). An antigen presenting cell is any cell capable of presenting an antigen to a T lymphocyte in the context of an MHC class II molecule. Thus B lymphocytes, mononuclear phagocytes (monocytes and macrophages) and dendritic cells are all considered to be APCs. However, the majority of nucleated cells are capable of acting as APCs under the appropriate conditions, e.g. when exposed to pro-inflammatory cytokines, and so the cell population may further comprise APCs which would not normally be regarded as mononuclear leukocytes.

The cell population comprises T cells which have previously encountered the antigen upon which the agent is based e.g. from a donor previously infected by, or sensitized to, an antigen or other antigen. This can be demonstrated by an appropriate assay that the T cell donor (which in vivo would be the agent recipient) has previously raised an immune response against the antigen; for example, the donor may be seropositive for the antigen, i.e. have circulating antibodies specific for the antigen. Under some circumstances the donor may not have circulating antibodies specific for the antigen, for example where insufficient time has elapsed since infection for detectable levels of antibodies to be raised, or where a substantial time has elapsed since exposure and antibody levels have fallen below the threshold of detectability. However, the term “seropositive” will be used throughout this specification to refer to any individual previously exposed to the relevant antigen, regardless of actual serological status, and the term “seronegative” should be construed accordingly, i.e as referring to an individual not previously exposed to the antigen.

Agents

The agent which comprises a peptide comprising at least one antigenic determinant of said antigen is capable of stimulating secretion of sCTLA-4 from T-cells activated by the previously encountered antigen. The agent will generally be a peptide having a sequence including at least one antigenic determinant of the antigen, and preferentially causes sCTLA-4 secretion preferentially over other responses as discussed above.

The peptide may comprise a plurality of contiguous antigenic determinants, which may be selected from immunodominant antigenic determinants or epitopes, and which may not be contiguous in the parent antigen. These may be selected to trigger the appropriate response in a plurality of tested individual seropositive ‘donors’ (see below)

The antigenic determinant may be a fragment of the parent antigen, howsoever obtained. However it will be appreciated that specific sequences might be altered to optimize their ability to induce sCTLA-4.

The term “peptide sequence” as used herein should not be taken to refer solely to a free peptide consisting essentially or exclusively of that sequence, although this is encompassed by the present invention. Without wishing to be bound by any particular theory, it is believed that the methods of the present invention are effective as long as the relevant sequence can be presented to T cells by antigen presenting cells within the population e.g. the peptide may be one capable of being processed and presented on MHC Class II molecules to T-cells (see e.g. FIG. 4). Typically such T cell epitopes will be at least 6 amino acids in length, more preferably at least 8 amino acids in length.

The ability of the peptide to act as a T cell epitope can be determined by assessing its ability to bind to the antigen binding groove of MHC II molecules. Peptide motifs which bind particular MHC alleles are known, and computer programs are available which can identify such motifs within protein sequences (Sturniolo. T., Bono. E., Ding. J., Raddrizzani. L., Tuereci. O., Sahin. U., Braxenthaler. M., Gallazzi. F., Protti. M. P., Sinigaglia. F., Hammer. J., Generation of tissue-specific and promiscuous HLA ligand database using DNA microarrays and virtual HLA class II matrices. Nat. Biotechnol. 17. 555-561(1999); Singh, H. and Raghava, G. P. S.(2001) ProPred: Prediction of HLA-DR binding sites. Bioinformatics,17(12), 1236-37)

The skilled person will be aware that any T cell that responds to a given peptide can also respond in a similar way to other peptides containing substitutions in residues that are not critical for MHC binding or T cell receptor recognition, and even to certain peptides that are substituted in critical residues. Such immunological cross reactivity of peptides can be demonstrated by showing that a particular T cell is capable of responding to more than one peptide. Such experiments may be performed using T cell clones. Techniques for cloning T cells are well known in the art. Without wishing to be bound by any particular theory, T cells of regulatory (Tr) phenotype may be implicated in the mechanism underlying the methods described herein. Such T cells do not proliferate significantly in response to stimulation, and suppress proliferation of other cells, and so can be difficult to clone. However, suitable techniques are known—see e.g. MacDonald, A J, Duffy, M, Brady, M T, McKiernan, S, Hall, W, Hegarty, J, Curry, M, and Mills K H G. CD4 T Helper Type 1 and Regulatory T Cells Induced against the Same Epitopes on the Core Protein in Hepatitis C Virus-Infected Persons. The Journal of Infectious Diseases (2002) 185:720-7.

Peptides derived from antigens as described herein, or identified using the methods herein, may be used to screen for immunologically cross reactive peptides which exert similar sCTLA-4 secretory effects by stimulating a similar or overlapping T cell population. Such cross reactive peptides may also be used in the present invention.

‘Variant’ peptides, and methods of producing peptides, are discussed in more detail hereinafter

The agents for use in the invention which are CD28 stimulatory binding agents, may be any known in the art, or which may be prepared by those skilled in the art on the basis of the disclosure herein. For example antibodies are well known which are stimulatory for the CD28 receptor (e.g. obtainable from Alexis Biochemicals, San Diego, USA via Axxora (UK)), and the agent may be all or part of such an antibody (for example which provides an appropriate CDR). As used herein the term “antibody” should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of Chimaeric antibodies are described in EP-A-0120694 and EP-A-0125023. It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the Vl and VH domains of a single antibody; (iv) the dAb fragment (Ward, E. S. et al., Nature 341, 544-546 (1989) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (W094/13804; P Holliger et al Proc. Natl. Acad. Sci. USA 90 6444-6448, 1993). Irrespective of the above, it will be appreciated that other agents may also employed in the present invention.

Where used herein (unless context demands otherwise) the term “sCTLA-4 stimulatory peptides” covers all of the peptide agents discussed above.

Assays

In another aspect, the present invention provides a method for providing agents as discussed above, the method comprising the steps of:

(i) contacting a cell population with said (putative) test agent,

(ii) determining whether sCTLA-4 secretion in said cell population is increased, and optionally

(iii) determining whether one or more pathogenic or otherwise undesirable T-cell activities is affected.

The sCTLA-4-based assays may be performed, in the light of the disclosure herein, using otherwise conventional T cell assays (see e.g. Devereux G, Hall Am and Barker R N. (2000) Measurement of T-helper cytokines secreted by cord blood mononuclear cells in response to allergens. J Immunol Methods. 234(1-2): 13-22; Mannering S I, Morris J S, Jensen K P, Purcell A W, Honeyman M C, van Endert P M and Harrison L C. (2003) A sensitive method for detecting proliferation of rare autoantigen-specific human T cells. J Immunol Methods 283: 173-183).

Thus, in a typical embodiment, peptides derived from a target protein antigen are added to a cell population (e.g. cultures of peripheral blood mononuclear cells (PBMC), lymphocytes, splenocytes or T cell subsets (e.g., CD4⁺ T cells, CD4⁺CD25⁺ T cells)

Preferred concentrations for peptides may range from 0.1 to 30 μg ml⁻¹, incubated for a period of between 3 to 7 days e.g. at 37° C. 5% CO2 in appropriate cell culture medium.

Following incubation relative levels of sCTLA-4 and other cytokines/soluble factors are assessed e.g. by the ELISA technique.

Proliferation of cells will be analyzed by tritiated thymidine incorporation, and cell division may be determined by CFSE incorporation followed by flow cytometry. Appropriate controls may include one or more of non-stimulated cells; cells stimulated with high affinity stimuli, including anti-CD3 and/or anti-CD28 monoclonal antibody; and mitogens including Concanavalin A and Staphylococcal enterotoxin B.

Sero-negative controls (healthy age- and sex-matched sero-negative donors) may be used to confirm that the effect of increased sCTLA-4 is a product of an antigen-specific immune response.

Preferred peptide agents which show at least 2, 3, 4, 5, 10, 20 or more enhancement of sCTLA-4 secretion (as compared with non-stimulated controls) while preferably giving less than this level of an activity as determined in (iii) may be selected. Preferably the peptide does not trigger a detectable pathogenic or otherwise undesirable T-cell activity in step (iii).

The (putative) agents, populations, activities and so on may be any of those described elsewhere herein.

The contacting step may include, for example, contacting a large (of the order of a 1 or 2 million, or more per ml of culture medium) population of PMC or T cells from individuals with the peptide. It may entail incubating the peptides and PMC for 1, 2, 3, 4, 5, 6, or 7 or more days at physiological temperature e.g. 37° C.

Selected peptides are then retested with a plurality of individual donors

Preferably the assay is performed on at least 5, 10, 15, 20, 25, 50 or more individual donor's PMC and peptides are selected which show the desired response in a plurality of these.

In another embodiment, the assay may be performed in the presence of putative modulators of the T cell response to identify those which can augment or enhance induction of sCTLA-4. In this case the assay is performed generally as above but using a peptide agent which is positive for enhanced sCTLA-4 secretion, and comparing its activity in the presence or absence of the putative modulator.

Following a positive outcome in the assays, peptide agents (or other agents), or modulators, may be formulated for use as medicaments, and thus the invention provides such processes for producing medicaments.

Preparations and Medicaments

The present invention also provides compositions of matter e.g. novel agents as discussed above, for example as obtained or obtainable from the assay. In all embodiments, the compositions may comprise ‘combination’ agents, which include CD28 stimulatory binding agents.

Preferably such agents are peptides which may be used in the treatment or prophylaxis of disease, which disease is characterized by a pathogenic immune or autoimmune response to an antigen, said peptide comprising at least one antigenic determinant of said antigen, and being capable of stimulating sCTLA-4 secretion by a population of T cells from an individual seropositive for the antigen (i.e. who may be symptomatic or asymptomatic for the disease).

As discussed above, the antigen is associated with the pathogenic immune or autoimmune response (i.e. a pathological lesion)—and the peptide may thus either be derived from the precise antigen targeted by pathogenic T-cells or from bystander self tissues damaged in consequence of that response.

Preferred agents are the agents of the invention as discussed above. Thus preferably the agent preferentially or selectively stimulates secretion of sCTLA-4 relative to a pathogenic or otherwise undesirable T-cell activity as discussed above. Preferred agents thereby inhibit the activity or activation of a T cell in response to the previously-encountered antigen as discussed above, which inhibitory effect may be assayed as described herein (see e.g. FIG. 7 and discussion thereof).

In a further aspect, the present invention provides a pharmaceutical composition comprising one or more sCTLA-4 stimulatory peptides as defined above and its use in methods of therapy or diagnosis (optionally in combination with an agent which is a CD28 stimulatory binding agent). In a further aspect, the present invention provides a pharmaceutical composition comprising a sCTLA-4 stimulatory peptide-encoding nucleic acid molecule and its use in methods of therapy or diagnosis (optionally in combination with a nucleic acid encoding an agent which is a CD28 stimulatory binding agent).

In further aspects, the present invention provides the above described sCTLA-4 stimulatory peptide sequences (optionally with CD28 stimulatory binding agents) and encoding nucleic acid molecules for use in the preparation of medicaments for therapy.

Pharmaceutical compositions of the present invention may comprise, in addition to the sCTLA-4 stimulatory peptide sequences and optionally CD28 stimulatory binding agents, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes (see below).

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included as required.

As the compositions of the present invention comprise peptides as active agents, they will typically be delivered by other routes, e.g. by intravenous, cutaneous or subcutaneous injection, or injection at the site of affliction, when the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

For delayed release, the active agents, e.g. sCTLA-4 stimulatory peptide sequences, may be included in a pharmaceutical composition for formulated for slow release, such as in microcapsules formed from biocompatible polymers or in liposomal carrier systems according to methods known in the art.

For continuous release of peptides, the peptides may be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of peptide therapeutics. Also useful, particularly for subdermal slow-release delivery, is a composition that includes a biodegradable polymer that is self-curing and that forms an implant in situ, after delivery in liquid form. Such a composition is described, for example in U.S. Pat. No. 5,278,202.

Methods of Treatment

In another aspect, the invention provides for use of the above agents in the treatment of (or preparation of a medicament for the treatment of) diseases characterized by a pathogenic T cell mediated immune or autoimmune response to an-antigen, for example disorders which may be improved by inhibition of CD28 and/or CTLA-4 interaction with CD80 and CD86.

In this respect, the preferred agents of the present invention, which stimulate endogenous sCTLA4 may have certain advantages over (for example) exogenous recombinant CTLA4. Firstly peptides are in principle less expensive to make than full length recombinant proteins. Secondly, they may have longer half-lives that immunoglobulin fusion proteins, which are cleared by cells expressing Fc receptors. Finally, the peptides share antigen-specificity with the pathogenic T-cells which are the target of treatment, rather than having a general immunosuppresive effect. Therefore their effect is focused at the site of action, while the remainder of the T-cell compartment is left intact.

Thus the present invention provides for peptide agents which induce the production of endogenous sCTLA-4 at the site of the pathological lesion. Such induced production of sCTLA-4 in lesions associated, for example, with autoimmune disease or graft rejection should quench T-cell mediated inflammatory responses with therapeutic benefits for the patient and little probability of undesired side-effects.

Such treatment may entail the administration of a prophylactically effective amount or a therapeutically effective amount of the peptide of the invention (optionally in combination with an agent which is a CD28 stimulatory binding agent) to subjects at risk of developing such diseases or to subjects already suffering from them.

For example the invention provides a method of treating a disease associated with undesirable T cell activation against an antigen, which method comprises administering to a patient suffering from said disease an sCTLA-4 secretion stimulating agent as described above.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Preferred diseases against which the present invention may be applied include any of the range of autoimmune diseases associated with pathogenic CD4⁺ T helper 1 T-cells (Th1) that co-ordinate persistent inflammatory responses against self tissues. Examples include autoimmune hemolytic anemia, type 1 diabetes, multiple sclerosis, and autoimmune thyroiditis. In transplantation, allo- or xeno-reactive T-cells specific for the graft could be suppressed. There is some evidence that T-cell suppression shortly after transplantation, maintained for a period of weeks, can lead to long term acceptance of the graft by the host, negating the need for lifelong immunosuppressant drug therapy.

Examples of auto-immune diseases in which specific antigens have been identified as potentially pathogenically significant include multiple sclerosis (myelin basic protein), insulin-dependent diabetes mellitus (glutamic acid decarboxylase), insulin-resistant diabetes mellitus (insulin receptor), rheumatoid arthritis, systemic lupus erythematosus, bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), and pernicious anaemia (intrinsic factor). Other indications include systemic lupus erythematosus (nucleosomal antigens) and Rheumatoid arthritis (Type II collagen). Thus these antigens, or particular fragments or epitopes thereof may be suitable target antigens.

Thus these antigens, or particular fragments or epitopes thereof may be suitable target antigens.

Allergic responses mediated by Th2 T-cells may also be targetted for suppression.

The target antigen may be an exogenous antigen which stimulates a response which also causes damage to host tissues. For example, acute rheumatic fever is caused by an antibody response to a Streptococcal antigen which cross-reacts with a cardiac muscle cell antigen. The target antigen may be one which provokes an atopic or allergic response, e.g. pollen (implicated in hayfever, e.g. Timothy Grass pollen), house dust mites (asthma), gliadin (coeliac disease), cosmetics, allergens administered via insect bites, nut allergens, or therapeutic products such as factor VIII, factor IX, blood group antigens, or monoclonal antibodies.

The methods of the present invention may be used to suppress responses to allogeneic or xenogeneic cells or tissues, including primary and secondary mixed lymphocyte reactions, graft rejection, and graft versus host disease.

Thus a subject intended to receive a cellular transplant may be given the transplant in conjunction with sCTLA-4 stimulatory peptides as described herein (optionally in combination with an agent which is a CD28 stimulatory binding agent) or nucleic acid encoding such peptide sequences (see below) in order to reduce the risk or degree of pathology in the recipient to those cells. In preferred embodiments, some or all of the cells to be transplanted may be engineered to express sCTLA-4 stimulatory peptides. Thus a cell to be transplanted may contain nucleic acid encoding a sCTLA-4 stimulatory peptide sequence according to the present invention such that the cell is capable of expressing the sCTLA-4 stimulatory peptide sequence (optionally in combination with an agent which is a CD28 stimulatory binding agent). The optimum methodology will depend on the identity of the cells to be engineered. Antigen presenting cells, e.g. dendritic cells, etc., may be engineered to express the sCTLA-4 stimulatory peptide sequence in such a manner that it is processed and presented in the context of the cells' own MHC II molecules. Other cell types may be engineered so that they secrete the expressed sequence, in order that it can be presented by neighboring APCS.

The test subject, or subject to be treated will typically be a mammal, and may be a human. In some embodiments, a test subject may be a non-human mammal e.g. a rodent, rabbit, etc. and will typically be seropositive for the antigen.

Modes of Administration

Agents of the present invention may be administered in any appropriate manner.

Peptides may preferably be administered by transdermal iontophoresis. One particularly useful means for delivering compounds is transdermal delivery. This form of delivery can be effected according to methods known in the art. Generally, transdermal delivery involves the use of a transdermal “patch” which allows for slow delivery of compound to a selected skin region. Such patches are generally used to provide systemic delivery of compound. Examples of transdermal patch delivery systems are provided by U.S. Pat. No. 4,655,766 (fluid-imbibing osmotically driven system), and U.S. Pat. No. 5,004,610 (rate controlled transdermal delivery system).

For transdermal delivery of peptides, transdermal delivery may preferably be carried out using iontophoretic methods, such as described in U.S. Pat. No. 5,032,109 (electrolytic transdermal delivery system), and in U.S. Pat. No. 5,314,502 (electrically powered iontophoretic delivery device).

For transdermal delivery, it may be desirable to include permeation enhancing substances, such as fat soluble substances (e.g., aliphatic carboxylic acids, aliphatic alcohols), or water soluble substances (e.g., alkane polyols such as ethylene glycol, 1,3-propanediol, glycerol, propylene glycol, and the like). In addition, as described in U.S. Pat. No. 5,362,497, a “super water-absorbent resin” may be added to transdermal formulations to further enhance transdermal delivery. Examples of such resins include, but are not limited to, polyacrylates, saponified vinyl acetate-acrylic acid ester copolymers, cross-linked polyvinyl alcohol-maleic anhydride copolymers, saponified polyacrylonitrile graft polymers, starch acrylic acid graft polymers, and the like. Such formulations may be provided as occluded dressings to the region of interest, or may be provided in one or more of the transdermal patch configurations described above.

In other treatment methods, the modulators may be given orally or by nasal insufflation, according to methods known in the art. For administration of peptides, it may be desirable to incorporate such peptides into microcapsules suitable for oral or nasal delivery, according to methods known in the art.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector. The vector could be targeted to the specific cells to be treated, or it could contain regulatory elements which are switched on more or less selectively by the target cells.

Use of nucleic acids in this way is considered to be applicable, mutatis mutandis, to any corresponding embodiment of the present invention in which administration of a peptide sequence is referred to. When the agent is a peptide, nucleic acids having appropriate coding sequences may likewise be administered instead. In related embodiments, cells may be contacted with peptides by contact with cells engineered to express the relevant peptides and either secrete them or present them in the context of MHC molecules.

A DNA expression vector encoding a peptide or protein antigen of interest is injected into the host animal, generally in the muscle or skin. The gene products are correctly glycosylated, folded and expressed by the host cell. The method is advantageous where the antigens are difficult to obtain in the desired purity, amount or correctly glycosylated form or when only the genetic sequences are known e.g. HCV. Typically, DNA is injected into muscles or delivered coated onto gold microparticles into the skin by a particle bombardment device, a “gene gun”.Genetic immunization has demonstrated induction of both a specific humoral but also a more broadly reacting cellular immune response in animal models of cancer, mycoplasma, TB, malaria, and many virus infections including influenza and HIV. See, for example, Mor et al. (1995) J Immunol 155:2039-46; Xu and Liew (1995) Immunology 84:173-6; and Davis et al. (1994) Vaccine 12:1503-9.

Ex-Vivo Embodiments

T-cell clones could be conditioned to secrete sCTLA-4 in the presence of their cognate antigen in vitro and then transferred into the patient.

For example peptide agents may be administered in vitro to a population of APCs. The population of APCs may then be contacted in vitro with a cell population comprising T cells from an individual. That cell population, or a subset thereof e.g. some or all of the T cells, may then be introduced into a test subject, or a subject to be treated, e.g. the subject from whom they were originally derived.

Alternatively, the population of APCs may be administered to a test subject, or a subject to be treated, e.g. the subject from whom they were originally derived. In this case contact between the cell population and the sCTLA-4 stimulatory peptide sequence takes place in vivo, via the APCs.

Thus cells or tissues may be removed from a donor individual or individual to be treated, treated with the sCTLA-4 stimulatory peptide sequence, and reintroduced to the donor. Suitable cells or tissues include particular type of antigen presenting cells, heterogeneous populations of cells, e.g. peripheral blood lymphocytes or subsets thereof, lymph nodes, etc.

As discussed above, preferably the cell population comprises at least T lymphocytes, preferably CD4⁺ T lymphocytes. More preferably, the cell population comprises at least T lymphocytes, preferably CD4⁺ T lymphocytes, and at least one type of APC. From the above description it can be seen that the cell population to be treated may in some embodiments be considered to comprise cells in situ in a test subject or subject to be treated.

Nucleic Acids and Methods of Making Peptides

In a further aspect, the present invention provides isolated nucleic acid molecules encoding the sCTLA-4 stimulatory sequences of the present invention (optionally in combination with an agent which is a CD28 stimulatory binding agent) e.g. for use in the methods discussed herein.

In further aspects, the present invention provides an expression vector comprising the above sCTLA-4 stimulatory sequence-encoding nucleic acid, operably linked to control sequences to direct its expression, as well as host cells transformed with the vectors. The present invention also includes a method of producing peptides of the preceding aspect, comprising culturing the host cells and isolating the sCTLA-4 stimulatory peptides thus produced.

In order to obtain expression of nucleic acids encoding sCTLA-4 stimulatory sequences, the sequences can be incorporated into a vector having control sequences operably linked to the encoding nucleic acid to control its expression. The vectors may include other sequences such as promoters or enhancers to drive the expression of the inserted nucleic acid, nucleic acid sequences so that the sCTLA-4 stimulatory sequence peptide is produced as a fusion, e.g. with one or more other such sCTLA-4 stimulatory sequences and/or nucleic acid encoding secretion signals so that the peptide produced in the host cell is secreted from the cell. Peptides/polypeptides/proteins can then be obtained by transforming the vectors into host cells in which the vector is functional, culturing the host cells so that the peptide is produced and recovering the peptide from the host cells or the surrounding medium. Prokaryotic and eukaryotic cells are used for this purpose in the art, including strains of E. coli, yeast, and eukaryotic cells such as COS or CHO cells.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, “Molecular Cloning: a Laboratory Manual”: 2nd edition, Sambrook et al., 1989, Cold Spring Harbor Laboratory. Press.

Cells and techniques may be selected such as to permit or enhance the folding and\or formation of disulphide bridges (see e.g. “Protein Folding” by R. Hermann, Pub. 1993, European Patent Office, The Hague, Netherlands, ISBN 90-9006173-8).

Peptides may be synthesized by any suitable method, such as by exclusively solid-phase techniques, by partial solid-phase techniques, by fragment condensation or by classical solution couplings. In conventional solution phase peptide synthesis, the peptide chain can be prepared by a series of coupling reactions in which the constituent amino acids are added to the growing peptide chain in the desired sequence.

Briefly, N-alpha-protected amino acid anhydrides are prepared in crystallized form or prepared freshly in solution and used for successive amino acid addition at the N-terminus. At each residue addition, the growing peptide (on a solid support) is acid treated to remove the N-alpha-protective group, washed several times to remove residual acid and to promote accessibility of the peptide terminus to the reaction medium. The peptide is then reacted with an activated N-protected amino acid symmetrical anhydride, and the solid support is washed. At each residue-addition step, the amino acid addition reaction may be repeated for a total of two or three separate addition reactions, to increase the percent of growing peptide molecules which are reacted. Typically, 1-2 reaction cycles are used for the first twelve residue additions, and 2-3 reaction cycles for the remaining residues.

The use of various N-protecting groups, various coupling reagents, e.g., dicyclohexylcarbodiimide or carbonyldiimidazole, various active esters, e.g., esters of N-hydroxyphthalimide or N-hydroxysuccinimide, and the various cleavage reagents, to carry out reaction in solution, with subsequent isolation and purification of intermediates, is well known classical peptide methodology. Classical solution synthesis is described in detail in the treatise “Methoden der Organischen Chemie (Houben-Weyl): Synthese von Peptiden”, E. Wunsch (editor) (1974) Georg Thieme Verlag, Stuttgart, W. Ger. Techniques of exclusively solid-phase synthesis are set forth in the textbook “Solid-Phase Peptide Synthesis”, Stewart & Young, Pierce Chemical-Co., Rockford, Ill., 1984, and are exemplified by the disclosure of U.S. Pat. No. 4,105,603. The fragment condensation method of synthesis is exemplified in U.S. Pat. No. 3,972,859. Other available syntheses are exemplified by U.S. Pat. Nos. 3,842,067 and 3,862,925.

Peptides are preferably prepared using the Merrifield solid phase synthesis, although other equivalent chemical syntheses known in the art can also be used as previously mentioned. Such solid-phase synthesis is commenced from the C-terminus of the peptide by coupling a protected alpha-amino acid to a suitable resin. Such a starting material can be prepared by attaching an alpha-amino-protected amino acid by an ester linkage to a chloromethylated resin or a hydroxymethyl resin, or by an amide bond to a benzhydrylamine (BHA) resin or paramethylbenzhydrylamine (MBHA) resin. The preparation of the hydroxymethyl resin is described by Bodansky et al., Chem. Ind. (London) 38, 1597-98 (1966). Chloromethylated resins are commercially available from Bio Rad Laboratories, Richmond, Calif. and from Lab. Systems, Inc. The preparation of such a resin is described by Stewart et al., “Solid Phase Peptide Synthesis”, supra.

The C-terminal amino acid, protected by Boc or Fmoc and by a side-chain protecting group, if appropriate, can be first coupled to a chloromethylated resin according to the procedure set forth in Chemistry Letters, K. Horiki et al. 165-168 (1978), using KF in DMF at about 60° C. for 24 hours with stirring, when a peptide having free acid at the C-terminus is to be synthesized.

Conditions for removal of specific alpha-amino protecting groups may be used as described in Schroder & Lubke, “The Peptides”, 1 pp 72-75, Academic Press (1965).

Activating reagents and their use in peptide coupling are described by Schroder & Lubke supra, in Chapter III and by Kapoor, J. Phar. Sci., 59, pp 1-27 (1970).

The success of the coupling reaction at each stage of the synthesis, if performed manually, is preferably monitored by the ninhydrin reaction, as described by E. Kaiser et al., Anal. Biochem. 34, 595 (1970). The coupling reactions can be performed automatically, as on a Beckman 990 automatic synthesizer, using a program such as that reported in Rivier et al. Biopolymers, 1978, 17, pp 1927-1938.

After completing the growing peptide chains, the protected peptide resin is treated with liquid hydrofluoric acid or trifluoroacetic acid (TFA) to deblock and release the peptides from the support. For preparing an amidated peptide, the resin support used in the synthesis is selected to supply a C-terminal amide, after peptide cleavage from the resin. After removal of the hydrogen fluoride, the peptide is extracted into 1M acetic acid solution and lyophilized.

The peptide can be isolated by an initial separation by gel filtration, to remove peptide dimers and higher molecular weight polymers, and also to remove undesired salts.

Variants

The sCTLA-4 stimulatory peptide sequences of the invention need not correspond exactly to the amino acid sequence of the pathogenic antigen. It is well known that proteins from wild type isolates of antigens often contain differences relative to the sequences of reference isolates of that agent. However, use of peptides synthesised according to reference sequences will typically provide the desired sCTLA-4 secretory effects.

It may be desirable deliberately to introduce sequence mutations relative to either a wild type isolate or reference isolate. For example, without wishing to be bound by any particular theory, it may be desirable to introduce mutations into a sCTLA-4 stimulatory peptide from a given antigen in order to enable it to bind to a broader range of MHC molecules.

Therefore sCTLA-4 stimulatory peptides may be used which differ from known or wild type antigenic determinant sequences for the corresponding region of the antigen, as long as they retain sufficient sCTLA-4 stimulatory capability. This can readily be determined by use of the methods of the present invention.

Variant peptides can be produced by a mixture of conservative variation, i.e. substitution of one hydrophobic residue such as isoleucine, valine, leucine or methionine for another, or the substitution of one-polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining peptide conformation. Also included are variants having non-conservative substitutions. As is well known to those skilled in the art, substitutions to regions of a peptide which are not critical in determining its conformation may not greatly affect its activity because they do not greatly alter the peptide's three dimensional structure, and so may not affect the desired activity, e.g. MHC binding. In regions which are critical in determining the peptides conformation or activity such changes may confer advantageous properties on the polypeptide. Indeed, changes such as those described above may confer slightly advantageous properties on the peptide e.g. altered stability.

Generally variant peptides may be extended at the N- or C-termini, and the C-terminus may be amidated or have a free acid form.

A peptide which is an amino acid sequence variant will generally share at least about 50%, 60%, 70%, 80%, 90% or more sequence identity with a wild type or reference sequence from the relevant antigen. In this connection, “sequence identity” means strict amino acid identity between the sequences being compared.

Once an amino acid substitution or other modification is made as described above, the peptide is screened for the requisite sCTLA-4 stimulatory activity, as described above.

Reduction of sCTLA-4 Secretion

The foregoing has been concerned with uses of the present invention in the context of increasing sCTLA-4 secretion, for example for the treatment of disease characterized by pathogenic T cell mediated immune or autoimmune response to an antigen.

However the present invention has application also in augmenting T cell mediated response in instances where it is desired to do so, for example where that response is desired therapeutically.

In such cases the agents of the invention are provided (e.g. screened as above) but selected where they show at least 2, 3, 4, 5, 10, 20 fold reduction of sCTLA-4 secretion as compared with non-stimulated cells, while still triggering the desirable T-cell activity in step (iii).

Analogously to W097/20574, such agents and methods may be useful where there is an inadequate T cell mediated response to an antigenic stimulus for an intended purpose, and where the response would be facilitated by reduced levels of endogenous sCTLA-4. In vivo T cell mediated responses include the generation of cytolytic T cells, Th1 T cells and the majority of antibody responses, particularly those involving class switching of immunoglobulin isotypes. The antigenic stimulus may be the presence of viral antigens on infected cells; parasitic or bacterial infection; or an immunization, e.g. vaccination, preparing monoclonal antibodies, etc.

Preferably, induced sCTLA-4 reduction could be used in the context of treatment against tumors, or pathogens that evade the host immune system by subverting the regulatory systems in place to prevent an active immune response.

Agents of this aspect of the present invention may also be used in conjunction with radiation and/or chemotherapeutic treatment which indirectly produces immune response stimulating agents. Such combined use can involve the simultaneous or sequential use of sCTLA-4 secretion inhibitors and an immune response stimulating agent.

Agents of this aspect of the present invention may be based on antigenic determinants from antigens which it is desired to target with a specific T cell response.

Preferred such antigens are tumor-specific antigens. Such antigens may be present in an abnormal context, at unusually high levels, or may be mutated forms. The tumor antigen may be administered with the subject blocking agents to increase the host T cell response against the tumor cells. Such antigen preparations may comprise purified protein, or lysates from tumor cells.

Examples of tumors antigens are cytokeratins, particularly cytokeratin 8, 18 and 19, as an antigen for carcinomas. Epithelial membrane antigen (EMA), human embryonic antigen (HEA-125); human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors.

Prostate specific antigen is an antigen of prostatic carcinomas, carcinoembryonic antigen of colon adenocarcinomas. HMB-45 is an antigen of melanomas. Chromagranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells.

Thus in this aspect the invention provides a method of inhibiting sCTLA-4 secretion by T cells which have previously been exposed to an antigen, which method comprises exposing said cells to an agent which inhibits endogenous secretion of sCTLA-4 therefrom, which agent is a peptide comprising at least one antigenic determinant of said antigen. Such methods may be used in to stimulate the activity e.g. of activated T cell.

In another aspect the invention provides a method of stimulating sCTLA-4 secretion by T cells, which method comprises exposing said cells to an agent which stimulates endogenous secretion of sCTLA-4 therefrom, which agent is a CD28 stimulatory binding agent. As demonstrated below, the inventors have shown that such agents can independently stimulate sCTLA-4, and may therefore also be used to inter alia increase tolerance to a particular target antigen. The preceding aspects of the invention, unless context demands otherwise, will this be understood to apply analogously to the use of CD28 stimulatory binding agents for stimulation of sCTLA-4 secretion by T cells.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. T-cells require two signals for activation. Signal 1 is a product of the T-cell receptor binding to a peptide antigen expressed by MHC Class II molecules. Only T-cells able to recognize the peptide are able to mount an immune response but these T-cells require a further signal (2) via ligation of CD28 with B7.1/B7.2 molecules on professional antigen presenting cells. Soon after activation CTLA-4 is transported to the cell surface where it competes with CD28 for ligation with B7.1/B7.2. As CTLA-4 binds with much higher affinity to the B7 molecules this interaction begins to dominate and an inhibitory signal is delivered via CTLA-4 to the T-cell.

FIG. 2. PBMC derived from an atopic donor secrete sCTLA-4 in response to the allergen Timothy grass. PBMC were incubated for 5 days at 37 C 5% CO₂ in the presence of Timothy grass (Th2), PPD (Th1) and LPS, and a negative control. ELISA was used to detect IL-4, IL-10 and sCTLA-4 while tritiated thymidine incorporation was used to detect proliferation.

FIG. 3. Differential stimulation of sCTLA-4 secretion by PBMC sampled from AIHA patients in response to incubation with RhD protein antigen.

FIG. 4. T-cells can be induced by peptide antigens derived from proteins to secrete a soluble form of sCTLA-4. The only known function of sCTLA-4 is to inhibit T-cell responses.

FIG. 5. The PPD recall antigen can boost production of sCTLA-4. PBMC from adults were incubated in the presence of the tuberculin PPD recall antigen or control preparations for 5 days at 37° C., 5% CO₂, and Th1/Th2 cytokine and sCTLA-4 levels were measured by ELISA. (a) A profile of an individual donor response to the PPD antigen compared with non-stimulated cells or non-specific stimulation with anti-CD3 monoclonal antibody. (b) In all individuals analyzed, PPD induced an increase in sCTLA-4 production (P<0.01, Wilcoxon) whereas, cord blood derived mononuclear cells that are naive to PPD did not (c). Thus sCTLA-4 enhancement requires antigen conditioning for function.

FIG. 6. Adsorption of sCTLA-4 from cell cultures. Anti-CTLA-4 antibody (10 mg ml⁻¹) was incubated in Hank's buffered saline solution (HBSS) for 2 hours at 37° C. in the presence of sterile plastic pins. The antibody pins were washed of free antibody and suspended in cell cultures for the duration of the experimental incubation period. This process removed approximately 85-90% of available sCTLA-4 and did not interfere with cell bound CTLA-4 processes. Soluble CTLA-4 bound to pins was detected using a modified ELISA.

FIG. 7a. Effect of CTLA-4 adsorption on T-cell proliferation and IFN-gamma production.

FIG. 7b. Effect of CTLA-4 blockade on cell divisional of CD4+ T-cells with PPD or anti-CD3 mAb compared with non-stimulated controls. PBMC were derived from a healthy volunteer donor, incubated with 0.2 μM CFSE for 5 minutes in darkness, washed and incubated with stimuli for 5 days at 37 C, 5% CO2 in the presence or absence of anti-CTLA-4 F (Ab′) 2 fragments at 0.5 μg ml-1. Flow cytometry was used to detect cell division (reduction in CFSE staining) and cells were counter-stained with CD4-PE stain. Cell activation status in each test sample was analyzed by staining the cells with CD25 and D69 (data not shown), as well as standard proliferation and cytokine ELISA assays as described above.

FIG. 8a. Stimulation of CD28 increases secretion of sCTLA-4 by human T-cells in the absence of PPD recall antigen (dark bars) and increases antigen-specific secretion of sCTLA-4 in the presence of PPD recall antigen (light bars). PBMC from two volunteer donors (top panels) were incubated with increasing amounts of soluble anti-CD28 stimulatory monoclonal antibody (0, 2 and 5 μg ml⁻¹) in the presence or absence of PPD recall antigen. Secretion of T cell lines specific for the Timothy Grass allergen (bottom panel), and polarised towards the Th1 (dark bars) or Th2 (light bars) phenotype, were also incubated with soluble anti-CD28 stimulatory monoclonal antibody (0-2 μg ml⁻¹).

FIG. 8b. Stimulation of CD28 induces sCTLA-4 in mouse lymphocytes. Murine lymphocytes were incubated with soluble anti-CD28 stimulatory monoclonal antibody (0-1 μg ml⁻¹) for 5 days. Cell proliferation, a marker of cellular activity, and sCTLA-4 secretion were compared.

FIG. 9. Results of peptide mapping assay to provide autoimmune hemolytic anemia (AIHA)) peptides which boost SCTLA-4 production.

### EXAMPLES

### Methods and Materials

Donors and sample preparation. Blood samples were taken from healthy volunteer donors after their consent was obtained. All adult donors confirmed that they had been immunized during childhood for tuberculosis, and were therefore likely to mount a recall response against Tuberculin purified protein derivative (PPD).

Cytokine Secretion ELISA. ELISA were based on previously published methods and were performed from 3-7 days of in vitro cell culture. The following antibody pairs were used: purified mouse anti-IFN-γ (clone NIB42) and biotinylated mouse anti-IFN-γ (clone 4S.B3); purified mouse anti-IL-10 (clone JES3-19F1) and biotinylated mouse anti-IL-10 (clone JES3-12G8); purified mouse anti-IL-4 (clone 8D4-8) and biotinylated mouse anti-IL-4 (clone MP4-25D2) (all from BD Biosciences, Oxford, UK). All recombinant human cytokines (IFN-γ, IL-4, and IL-10) were purchased from Peprotech EC Ltd. (London, UK).

We also developed a similar secretion ELISA for sCTLA-4 with antibodies identified previously. Briefly, sCTLA-4 was measured in vitro by incubating a capture anti-CTLA-4 antibody (clone: BNI3 2μg ml⁻¹) in 96 well Nunc Maxisorp plates for 2 hours at 37° C. Plates were washed and blocked with 3% BSA before the addition of test cell culture suspension. Plates were incubated overnight at 37° C., 5% CO₂, washed and further incubated with a biotinylated anti-CTLA-4 antibody (Clone: AS32; Antibody solutions, Mountain View, Calif., USA) for 2 hours at RT in darkness. The plates were washed again and incubated with streptavidin-labelled alkaline phosphatase (Sigma Aldrich Ltd., Poole, Dorset) for 1 hour at RT in darkness. After a final wash, plates were incubated with Phosphatase substrate (Sigma Aldrich) at RT in darkness. Developed plates were measured with a Multiskan MS microplate photometer (Life and Laboratory Sciences, Basingstoke, UK) at an absorbance of 405 nm. A standard curve was constructed using CTLA4-Ig (Alexis Biochemicals, Nottingham, UK) doubly diluted from 8000 μg ml⁻¹ and incubated on the capture antibody coated plates precisely as described for cell suspensions above.

Proliferation Assay. Cell proliferation was assessed by the incorporation of ³H thymidine, as previously described.

Enrichment or depletion of T-cell subsets. CD4⁺ T-cells were fractionated with a Dynal® CD4⁺ isolation kit (Dynal Biotech, Wirral, UK). CD4⁺ Th2 T-cells were isolated with an anti-CRTH2 Th2 T-cell isolation kit, while CD4⁺CD25⁺ were either enriched or depleted with a regulatory T-cell negative isolation kit (both Miltenyi Biotec Ltd., Bisley, UK).

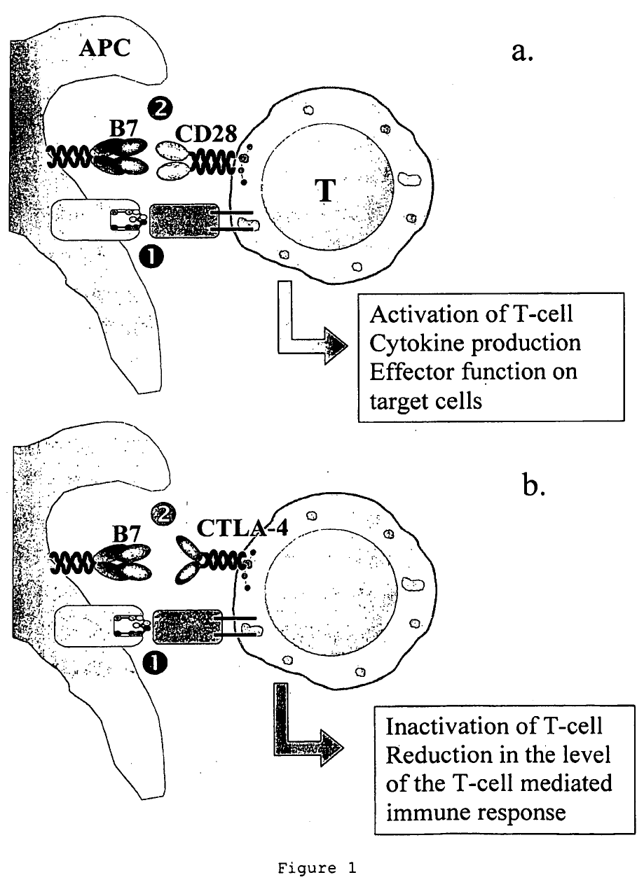

Adsorption of sCTLA-4 in vitro. Soluble CTLA-4 was depleted during cell culture using the following method. Anti-CTLA-4 capture antibody was incubated with plastic “pins” (Perbio Science UK Ltd., Cramlington, Northumberland, UK. Note: Pins are “blank” versions of plastic pins used for peptide synthesis) in 0.2% BSA for 2 hours, washed three times with Hank's Balanced salt solution, and suspended in wells containing cell cultures such that there was no contact between the pins and the cells. At the end of the experiment pins were removed from cell cultures, blocked with 3% BSA, and a modified ELISA performed to detect adsorbed pin-bound sCTLA-4 using the same reagents described above (see FIG. 1).

Antibodies and antigens. Stimulatory anti-CD28 antibody (clone ANC28.1/5D10) and anti-CTLA-4 F(ab′)2 fragments for CTLA-4 blockade (clone ANC152.2/8H5) were obtained from Alexis Biochemicals, while a murine IgG control antibody was purchased from Serotec Ltd. (Oxford, UK). Tuberculin purified protein derivative was purchased from Statens Serum Institut (Copenhagen, Denmark) and dialysed overnight before use at 5 μg ml⁻¹. Stimulatory anti-CD3 antibody (clone OKT3) was purified from hybridoma cell culture supernatants against protein A and added to cultures at 5 μg ml⁻¹. Concanavalin A (Sigma Aldrich) was added to cell cultures at 2 μg ml⁻¹.

Statistical analysis Differences in sCTLA-4 concentration between treatments was analyzed using the Wilcoxon Matched-Pairs Signed-Ranks Test.

### Example 1

### Detection Of Increased Levels Of sCTLA-4 Following Incubation With The PPD Antigen.

Previous studies of sCTLA-4 secretion by T-cells demonstrated that resting T-cells secrete sCTLA-4, and upon activation with anti-CD3 antibody, reduce sCTLA-4 secretion corresponding with a switch to increased expression of full-length CTLA-4.

We analyzed responses to a typical recall antigen, PPD, and examined whether after incubation with PPD, PBMC increased secretion of sCTLA-4 compared with non-stimulated cells isolated from healthy volunteer donors.

In this in vitro system, levels of sCTLA-4 in non-stimulated cell cultures varied between individuals and ranged from approximately 150 to around 10,000 pg ml⁻¹.

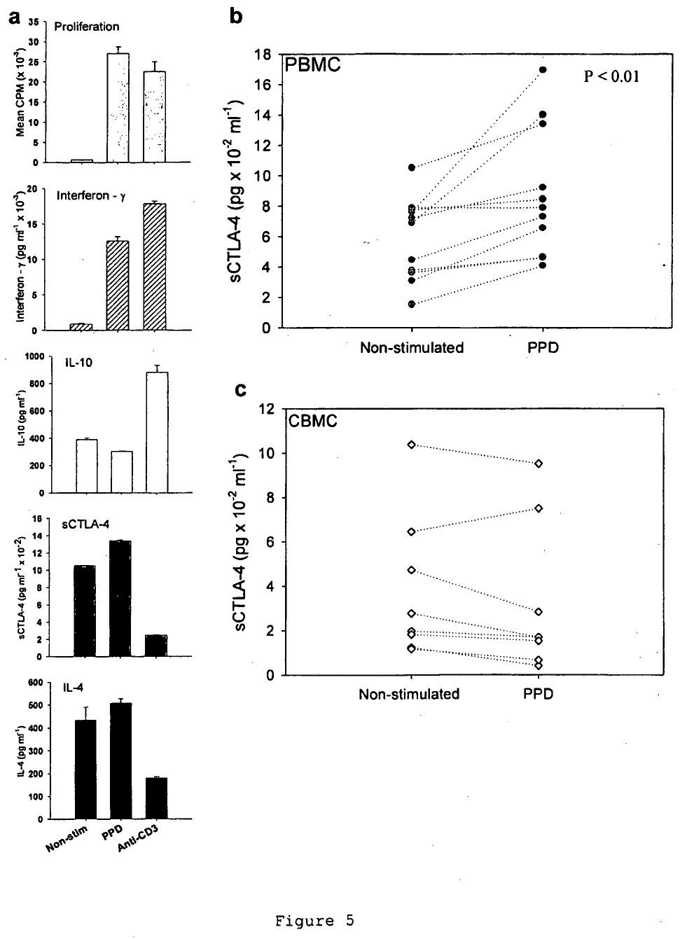

FIG. 5a illustrates a comparison of cytokine secretion patterns between non-stimulated PBMC and PBMC incubated either with PPD or anti-CD3 antibody. As described previously, sCTLA-4 levels in vitro are dramatically reduced compared with non-stimulated PBMC (Magistrelli G, Jeannin P, Herbault N, Benoit De Coignac A, Gauchat J F, Bonnefoy J Y and Delneste Y. (1999) A soluble form of CTLA-4 generated by alternative splicing is expressed by nonstimulated human T cells. Eur J. Immunol. 29(11): 3596-3602; Oaks M K, Hallett K M, Penwell R T, Stauber E C, Warren S J and Tector A J. (2000) A native soluble form of CTLA-4. Cell Immunol. 201(2): 144-153.), but in contrast, sCTLA-4 levels increased in vitro following PPD incubation. Proliferation and IFN-γ secretion appear similar between PPD and anti-CD3 cultures but there is an increase in IL-10 mediated by anti-CD3 compared with PPD. Further, analysis of SCTLA-4 levels in 12 healthy individuals revealed that while anti-CD3 invariably reduced levels of sCTLA-4 secretion (data not shown), PPD almost invariably raised sCTLA-4 levels compared with non-stimulated PBMC (FIG. 5b, P<0.01).

Co-analysis of a correlation between either IFN-γ, IL-10, or IL-4 supernatant concentration and sCTLA-4 following PPD incubation, revealed an inverse correlation between IFN-γ and sCTLA-4 (FIG. 5d, r2=0.52, P<0.01)

### Example 2

### Adsorption Of sCTLA-4 Increases IFN-γ and Proliferation

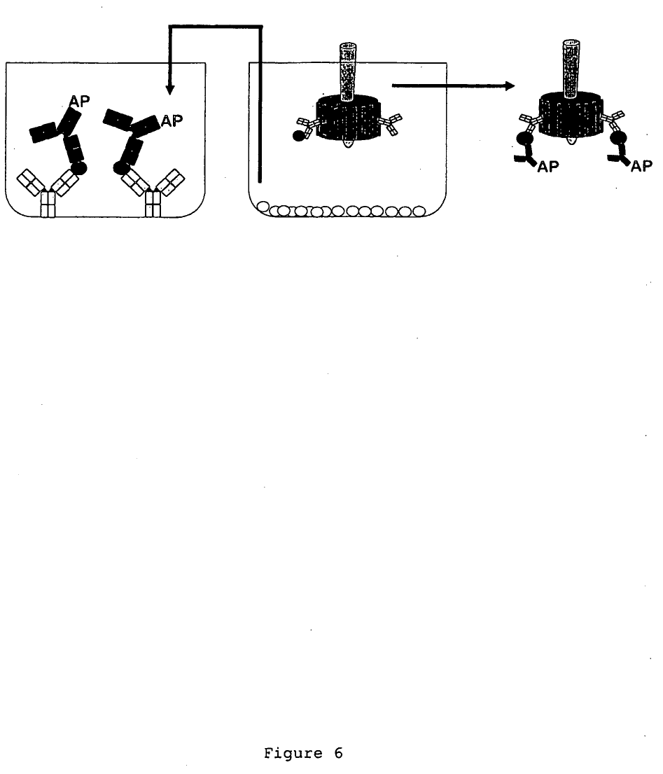

As PPD enhanced production of sCTLA-4 by PBMC from healthy adult donors compared with non-stimulated cells, we wished to determine whether adsorption of sCTLA-4 from cell cultures enhanced cell proliferation and the production of the Th1 T-cell cytokine, IFN-γ. To do this we coated sterile plastic pins with either purified anti-CTLA-4 antibody or mouse isotype control immunoglobulin (FIG. 6).

A dose range experiment determined that while anti-CTLA-4 antibody on a single pin at 1 μg ml⁻¹ was sufficient to adsorb detectable amounts of sCTLA-4, a concentration of 10 μg ml⁻¹ depleted at least 80% of available sCTLA-4 over a period of five days. In later experiments duplicate pins per well were used.

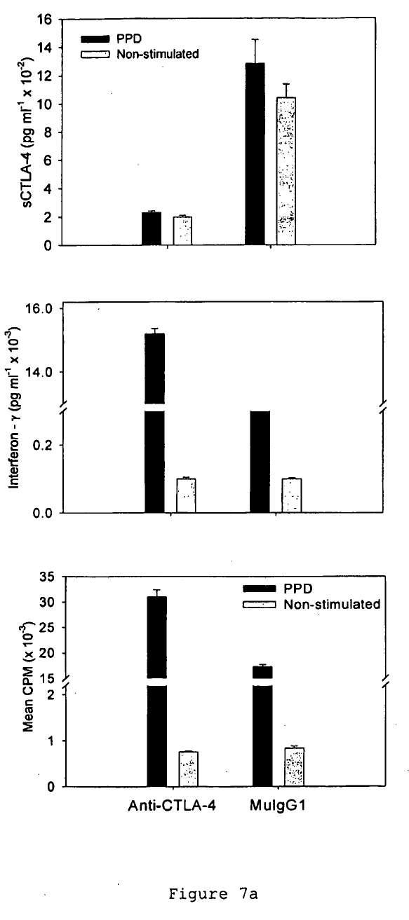

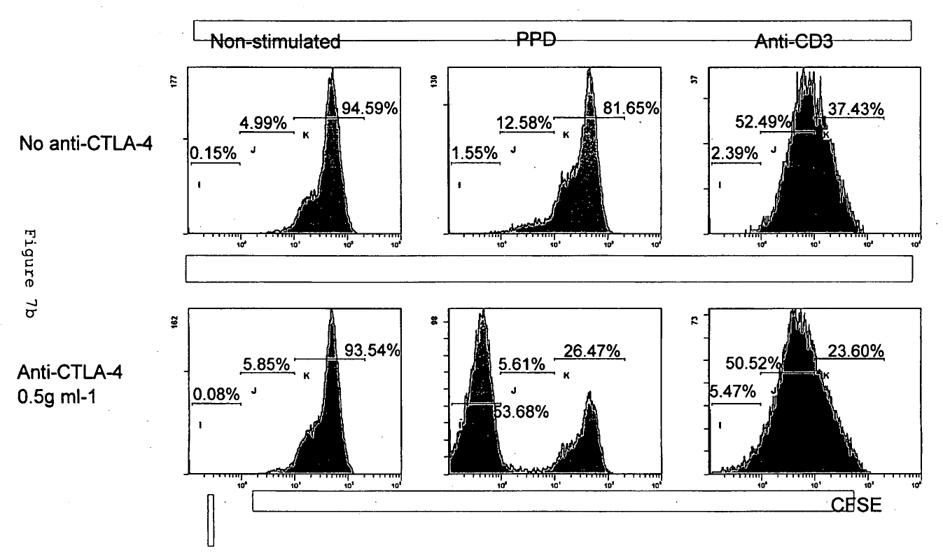

Analysis of PBMC cultures depleted of sCTLA-4 (FIG. 7a) confirmed good depletion of sCTLA-4, and a modest but significant (P<0.01) increase in both IFN-γ concentration and proliferation in response to PPD. Thus depletion of sCTLA-4 appears to amplify the immune response against the PPD antigen. Interestingly, in each sample tested there was no significant effect of sCTLA-4 depletion on non-stimulated cells suggesting that sCTLA-4 is not functioning simply to contain the activity of T-cells per se.

To analyze the effect of sCTLA-4 adsorption further, PBMC were infused with 0.2 μM CFSE before stimulation with antigen, and incubation with antibody-coated pins (FIG. 7b).

### Example 3

### Analysis Of PPD-Specific sCTLA-4 Secretion By T-Cell Subsets. Th2 T-Cells/CD4+CD25+ IL-10+

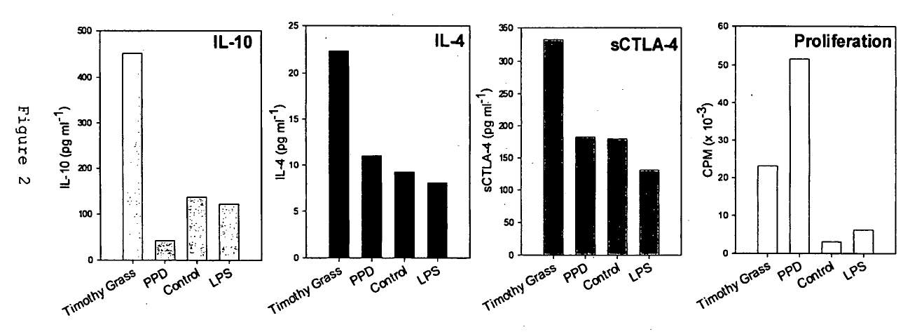

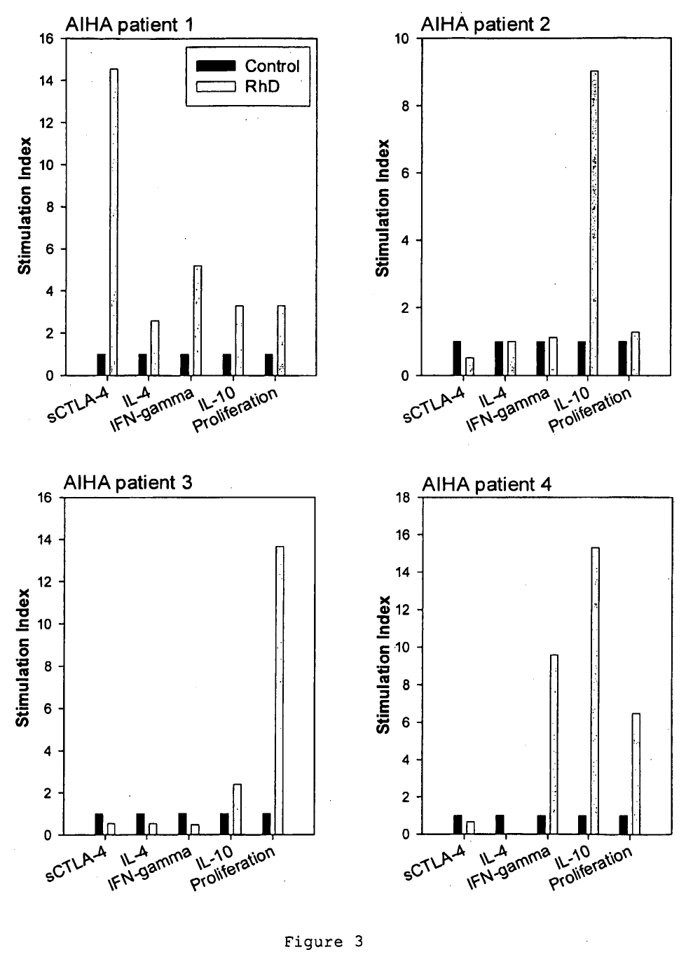

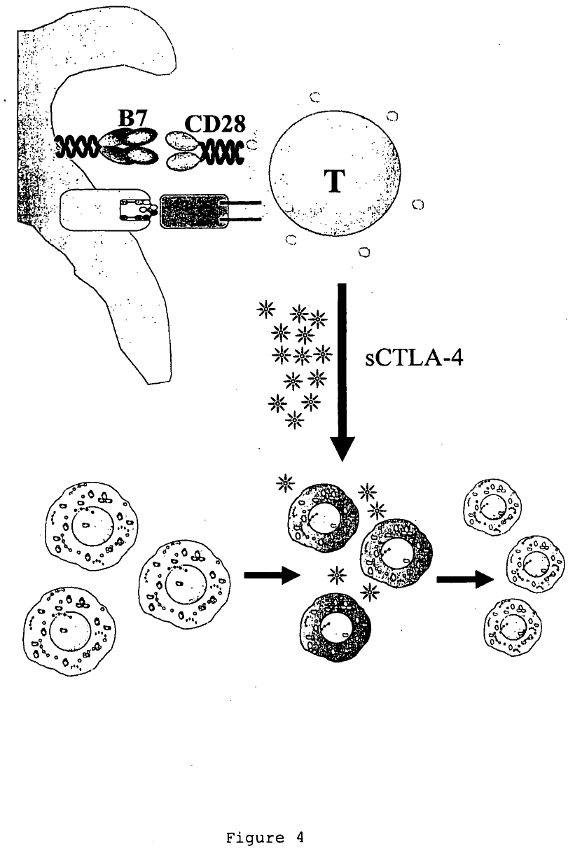

Primary antigen-specific Th2 responses, characterized by IL-4 production, correspond with an increase in sCTLA-4 whereas Th1 responses result in reduced sCTLA-4 secretion (FIG. 2). PBMC from patients with AIHA (a Th1 mediated autoimmune disease), respond specifically to the AIHA-associated autoantigen, RhD, but the cytokine profile associated with that response varies between individuals. From an initial sample of six we have identified one patient whose PBMC respond to the RhD autoantigen by secreting higher levels of sCTLA-4 and IL-4 compared with negative controls (FIG. 3).

### Example 4

### CD28

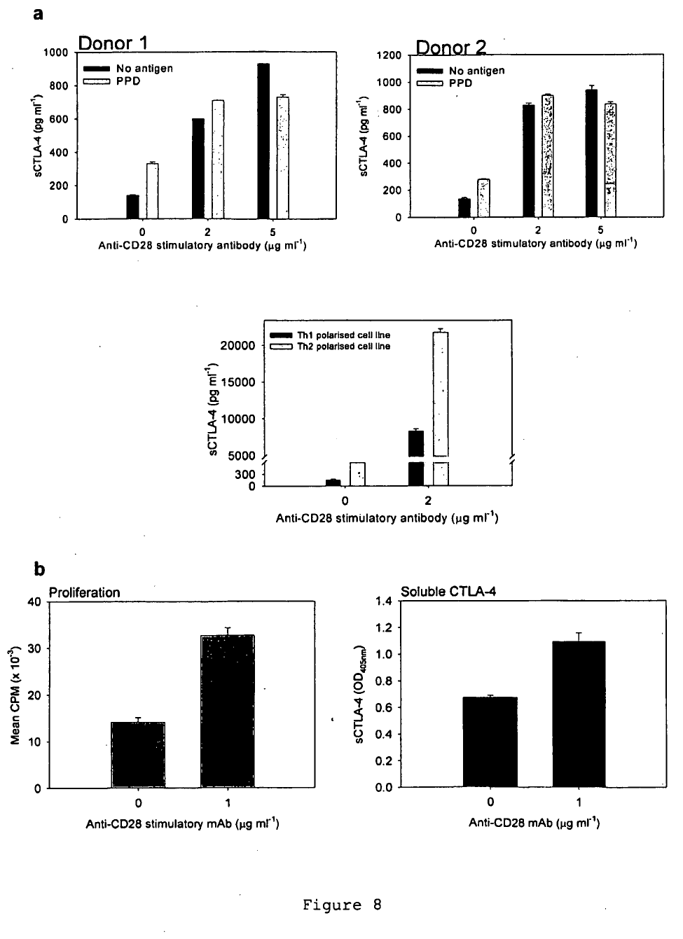

As shown in FIG. 8a, anti-CD28 stimulation of human PBMC over a dose range of 0-5 μg ml⁻¹ increased secretion of sCTLA-4 in the absence of PPD recall antigen. Furthermore, incubation of T cell lines polarised towards either the Th1 or the Th2 phenotype with 2 μg ml⁻¹ anti-CD28 stimulatory antibody also increased sCTLA-4 secretion.

FIG. 8a also shows that incubation of human PBMC with 2 or 5 μg ml⁻¹ anti-CD28 stimulatory antibody in the presence of PPD recall antigen increases antigen-specific sCTLA-4 secretion.

Incubation of mouse lymphocytes with 1 μg ml⁻¹ anti-CD28 stimulatory antibody increases lymphocyte activity and promotes sCTLA-4 secretion (see FIG. 8b).

### Example 5

### Peptides

The following example demonstrates how for a given indication (here, autoimmune hemolytic anemia (AIHA)) peptides which boost sCTLA-4 production can be provided in accordance with the methods described herein.

Peptide mapping experiments to identify peptides which stimulate sCTLA-4 production were performed as follows. Each peptide (1 to 42) was derived from the RhD autoantigen. Lymphocytes (1 million per peptide) from patients with autoimmune haemolytic anaemia were added to individual peptides and incubated for five days at 37° C. At the end of this period the cell cultures were assessed for an increase in sCTLA-4 production. In these assays a stimulation index of 2 is considered a positive result (twice as much sCTLA-4 detected compared with non-stimulated control).

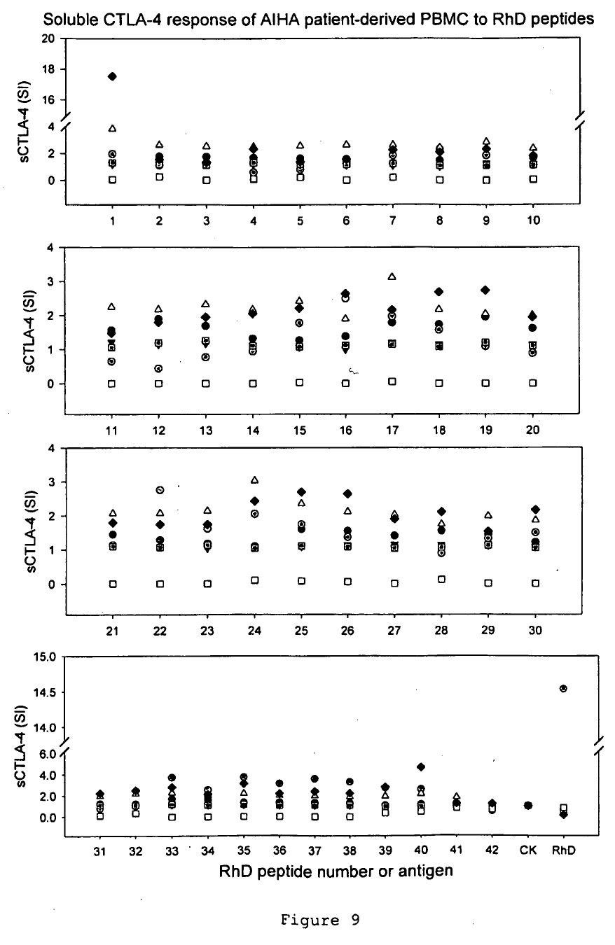

The results are shown in FIG. 9. As can be seen there are seven symbols for each peptide—each symbol represents a different AIHA patient, i.e., the experiment was repeated seven times with a different AIHA patient each time. Certain peptides in some cases stimulated sCTLA-4 production by three, four or even ten times higher than non-stimulated cells alone.

Peptide 1 [SSKYPRSVRRCLPLW (residues 2-16)], was particularly effective, although peptides 16,17,22,24,25,26,33,35,and 40 were also of interest:

16: NLRMVISNIFNTDYH
17: NTDYHHMNMMHIYVFA
22: GALFLWIFWPSFNSA
24: IERKNAVFNTYYAVA
25: YYAVAVSVVTAISGS
26: AISGSSLAHPQGKIS
33: IPHSSIMGYNFSLLG
35: IYIVLLVLDTVGAGN
40: IWKAPHEAKYFDDQV

1: A method of stimulating soluble cytotoxic T-lymphocyte antigen-4 (sCTLA-4) secretion by T cells, which method comprises exposing said cells to a stimulatory agent such as to induce secretion of endogenous sCTLA-4 therefrom.

2: A method of stimulating soluble cytotoxic T-lymphocyte antigen-4 (sCTLA-4) secretion by T cells which have previously been exposed to an antigen, which method comprises exposing said cells to an agent which stimulates endogenous secretion of sCTLA-4 therefrom, which agent is a peptide comprising at least one antigenic determinant of said antigen.

3: A method as claimed in claim 2 comprising exposing said cells to a combination of:
(i) an agent which comprises a peptide comprising at least one antigenic determinant of said antigen, and
(ii) a CD28 stimulatory binding agent.

4: A method as claimed in claim 2 wherein the antigen is associated with a pathogenic immune or autoimmune response.

5: A method as claimed in claim 2 whereby the activity or activation of the T cells in response to the antigen is inhibited.

6: A method as claimed in claim 1 wherein the peptide comprises a plurality of antigen determinants.

7: A method for providing an agent capable of stimulating soluble cytotoxic T-lymphocyte antigen-4 (sCTLA-4) secretion by T cells, the method comprising the steps of:
(i) contacting a cell population with a putative test agent, and
(ii) determining whether sCTLA-4 secretion in said cell population is increased.

8: A method as claimed in claim 7 wherein the putative test agent is a peptide comprising at least antigenic determinant of an antigen for which the individual from which the T cells are derived is seropositive.

9: A method as claimed in claim 7 wherein the putative test agent comprises a putative modulator of the T cell response and a peptide agent which is capable of enhancing sCTLA-4 secretion.

10: A method as claimed claim 7 further comprising the step of formulating the agent as a medicament.

11: A composition comprising a peptide comprising at least one antigenic determinant of an antigen, and being capable of stimulating sCTLA-4 secretion by a population of T cells from an individual seropositive for the antigen, for use in the treatment or prophylaxis of a disease, which disease is characterized by a pathogenic immune or autoimmune response to the antigen.

12: A composition as claimed in claim 11 wherein the composition further comprises a CD28 stimulatory binding agent.

13: A composition as claimed in claim 11 wherein the peptide comprises a plurality of antigen determinants.

14: A composition comprising a nucleic acid encoding a peptide comprising at least one antigenic determinant of an antigen, which peptide is capable of stimulating sCTLA-4 secretion by a population of T cells from an individual seropositive for the antigen, for use in the treatment or prophylaxis of a disease, which disease is characterized by a pathogenic immune or autoimmune response to the antigen.

15: A composition as claimed in claim 14, wherein the composition further comprises a nucleic acid encoding an agent which is a CD28 stimulatory binding agent.

16: A method for the treatment or prophylaxis of a disease comprising administering a composition as claimed in claim 11, wherein the disease is characterized by a pathogenic immune or autoimmune response to the antigen.

17: A A method as claimed in claim 16 wherein the disease and antigen respectively are selected from the group consisting of:
(i) multiple sclerosis and myelin basic protein;
(ii) insulin-dependent diabetes mellitus and glutamic acid decarboxylase;
(iii) insulin-resistant diabetes mellitus and insulin receptor,;
(iv) rheumatoid arthritis or systemic lupus erythematosus or bullous pemphigoid and collagen type XVII;
(v) autoimmune haemolytic anaemia and Rh protein;
(vi) auto-immune thrombocytopenia and GpIIb/IIIa;
(vii) myasthenia gravis and acetylcholine receptor;
(viii) Graves' disease and thyroid-stimulating hormone receptor;
(ix) glomerulonephritis and alpha3(IV)NCl collagen;
(x) pernicious anaemia and intrinsic factor;
(xi) systemic lupus erythematosus and nucleosomal antigens, and
(xii) rheumatoid arthritis and collagen type II.

18: A method as claimed in claim 16 wherein the antigen is an exogenous antigen which stimulates a response which also causes damage to host tissues.

19: A method as claimed in claim 18 wherein the disease and antigen respectively are selected from the group consisting of:
(i) acute rheumatic fever and a Streptococcal antigen;
(ii) hayfever and a pollen antigen;
(iii) asthma and a house dust mite antigen; and
(iv) celiac disease and gliadin.

20: A method as claimed in claim 19 wherein the source of antigen is an allergen selected from: a cosmetic; an insect bite; a nut allergen; and a therapeutic product.

21: A method as claimed in claim 16 wherein the pathogenic immune or autoimmune response is to allogeneic or xenogeneic cells or tissues.

22: A method as claimed in claim 21 wherein the treatment or prophylaxis comprises providing the composition to a subject intended to receive a cellular transplant, wherein the composition is provided in conjunction with the cellular transplant in order to reduce the risk or degree of pathology in the subject.

23: A method of inhibiting sCTLA-1 secretion by T cells which have previously been exposed to an antigen, which method comprises exposing said cells to an agent which inhibits endogenous secretion of sCTLA-4 therefrom, which agent is a peptide comprising at least one antigenic determinant of said antigen.

24: A method as claimed in claim 23 which is used to stimulate the activity of an activated T cell against the antigen.

25: A method as claimed in claim 24 wherein the antigen is a tumor-specific antigen.

26: A method as claimed in claim 1 wherein the agent is a CD28 stimulatory binding agent.

27: The method of claim 7 further comprising determining whether one or more pathogenic or otherwise undesirable T-cell activities in affected.

28: A method for the treatment or prophylaxis of a disease comprising administering a composition as claimed in claim 14, wherein the disease is characterized by a pathogenic immune or autoimmune response to the antigen.

29: A method as claimed in claim 28 wherein the disease and antigen respectively are selected from the group consisting of:
(i) multiple sclerosis and myelin basic protein;
(ii) insulin-dependent diabetes mellitus and glutamic acid decarboxylase;
(iii) insulin-resistant diabetes mellitus and insulin receptor;
(iv) rheumatoid arthritis or systemic lupus erythematosus or bullous pemphigoid and collagen type XVII;
(v) autoimmune haemolytic anaemia and Rh protein;
(vi) auto-immune thrombocytopenia and GpIIb/IIIa;
(vii) myasthenia gravis and acetylcholine receptor;
(viii) Graves' disease and thyroid-stimulating hormone receptor;
(ix) glomerulonephritis and alpha3(IV)NCl collagen;
(x) pernicious anaemia and intrinsic factor;
(xi) systemic lupus erythematosus and nucleosomal antigens; and
(xii) rheumatoid arthritis and collagen type II.

30: A method as claimed in claim 28 wherein the antigen is an exogenous antigen which stimulates a response which also causes damage to host tissues.

31: A method as claimed in claim 30 wherein the disease and antigen respectively are selected from the group consisting of:
(i) acute rheumatic fever and a Streptococcal antigen;
(ii) hayfever and a pollen antigen;
(iii) asthma and a house dust mite antigen; and
(iv) celiac disease and gliadin.

32: A method as claimed in claim 31 wherein the source of antigen is an allergen selected from: a cosmetic; an insect bite; a nut allergen; and a therapeutic product.

33: A method as claimed in claim 28 wherein the pathogenic immune or autoimmune response is to allogeneic or xenogeneic cells or tissues.

34: A method as claimed in claim 33 wherein the treatment or prophylaxis comprises providing the composition to a subject intended to receive a cellular transplant, wherein the composition is provided in conjunction with the cellular transplant in order to reduce the risk or degree of pathology in the subject.


SEQUENCE LISTING (10 sequences)

SEQ ID NO: 1 - protein, 15 aa, Artificial sequence
  Peptide 1 derived from the RhD autoantigen
SSKYPRSVRRCLPLW

SEQ ID NO: 2 - protein, 15 aa, Artificial sequence
  Peptide 16 derived from the RhD autoantigen
NLRMVISNIFNTDYH

SEQ ID NO: 3 - protein, 16 aa, Artificial sequence
  Peptide 17 derived from the RhD autoantigen
NTDYHHMNMMHIYVFA

SEQ ID NO: 4 - protein, 15 aa, Artificial sequence
  Peptide 22 derived from the RhD autoantigen
GALFLWIFWPSFNSA

SEQ ID NO: 5 - protein, 15 aa, Artificial sequence
  Peptide 24 derived from the RhD autoantigen
IERKNAVFNTYYAVA

SEQ ID NO: 6 - protein, 15 aa, Artificial sequence
  Peptide 25 derived from the RhD autoantigen
YYAVAVSVVTAISGS

SEQ ID NO: 7 - protein, 15 aa, Artificial sequence
  Peptide 26 derived from the RhD autoantigen
AISGSSLAHPQGKIS

SEQ ID NO: 8 - protein, 15 aa, Artificial sequence
  Peptide 33 derived from the RhD autoantigen
IPHSSIMGYNFSLLG

SEQ ID NO: 9 - protein, 15 aa, Artificial sequence
  Peptide 35 derived from the RhD autoantigen
IYIVLLVLDTVGAGN

SEQ ID NO: 10 - protein, 15 aa, Artificial sequence
  Peptide 40 derived from the RhD autoantigen
IWKAPHEAKYFDDQV